(19) United States
(12) Patent Application Publication
(10) Pub. No.: US 2022/0211436 A1
(43) Pub. Date: Jul. 7, 2022

(54) METHODS AND APPARATUS FOR HIGH-SPEED AND HIGH-ASPECT RATIO LASER SUBTRACTIVE MATERIAL PROCESSING

(71) Applicants: Board of Regents, The University of Texas System, Austin, TX (US); Research Development Foundation, Carson City, NV (US)

(72) Inventors: Thomas E. MILNER, Austin, TX (US); Nitesh KATTA, Austin, TX (US)

(73) Assignees: Board of Regents, The University of Texas System, Austin, TX (US); Research Development Foundation, Carson City, NV (US)

(21) Appl. No.: 17/611,315

(22) PCT Filed: May 12, 2020
(86) PCT No.: PCT/US2020/032443

§ 371 Date: Nov. 15, 2021

Related U.S. Application Data
(60) Provisional application No. 62/847,577, filed on May 14, 2019.

(51) Int. Cl. A61B 18/20

(52) U.S. Cl. CPC A61B 18/203; A61B 2018/00005

(57) ABSTRACT

Exemplary embodiments of the present disclosure apparatus and methods that provide for subtractive material processing, including efficient and precise ablation of tissues. Certain embodiments include a first laser configured to direct a first pulse of energy at a first wavelength to a region of tissue; a second laser configured to direct a second pulse of energy at a second wavelength to the region of tissue; and a control system configured to control operation of the first laser and the second laser.

### CROSS-REFERENCE TO RELATED APPLICATIONS

This application claims priority to U.S. Provisional Patent Application Ser. No. 62/847,577 filed May 14, 2019, the entire contents of which are incorporated herein by reference.

### BACKGROUND INFORMATION

High energy light sources including, for example, lasers are commonly used in apparatus and methods for tissue ablation in medical procedures. Such systems often do not provide acceptable levels of tissue removal rate, precision in cutting and minimization of non-specific residual damage.

In typical existing systems, femtosecond and picosecond pulse duration lasers have been considered where an emphasis is placed on high precision in tissue removal. However, the tissue removal rate in such systems can be unsatisfactorily low. If higher tissue removal rates are desired, precision of removal and non-specific residual damage can be sacrificed.

Conversely, in systems that place an emphasis on higher tissue removal rates, the accuracy and precision of the tissue removal may be less than satisfactory. The increased energy associated with such systems can reduce the ability of the user to precisely control the application and lead to damage of surrounding tissue.

Accordingly, systems and methods are desired that overcome these and other limitations associated with existing systems and methods.

### SUMMARY

Exemplary embodiments of the present disclosure include systems and methods for rapid and precise ablation of tissues.

Lasers have long been applied for subtractive material processing. Laser subtractive processes begin with spatially/temporally preparing the beam and directing the beam from the environment onto a material intended for subtractive processing. At some depth in the material after the incident laser radiation has passed through the environment-material interface, the laser radiation has sufficient pulsed fluence rate and/or pulse fluence energy to induce a phase change (solid to gas, solid to plasma, solid/liquid to gas or solid/liquid to plasma) in the material and form a bubble. The bubble may contain a high temperature gas or a plasma or a combination (gas and plasma) and develop a transient pressure that is larger than that in the surrounding material and/or environment that can lead to shockwave generation and bubble expansion.

In the course of the development of a tissue surgical laser system, the inventors constructed a thulium (Tm) laser system for rapid tissue cutting. During development, the inventors noted a limitation in using Tm lasers to cut tissue. The inventors applied the Tm laser radiation to ex vivo and in vivo tissues and observed the bubble-formation and expansion process using optical coherence tomography (OCT). The inventors observed that the tissue removal rate efficiency was inconsistent and did not always provide a clean and uniform cut in the tissue. The Tm laser was originally selected, to meet the conflicting requirements of rapid and precise ablation. OCT imaging of the light-tissue interaction revealed that when pulsed Tm laser light enters the tissue a rapidly expanding vapor bubble is formed. It is understood the OCT imaging incorporated in the disclosed system is optional, and that other embodiments of the present disclosure may not utilize OCT imaging.

It is believed the reason for the inconsistent Tm laser cutting efficiency is due to how the rapidly expanding vapor bubble evolves in time. If the vapor bubble can provide a sufficient shear force on the most superficial layer of the tissue, then the bubble will tear the tissue surface and clean laser cutting is achieved. If the expanding/collapsing bubble cannot provide sufficient shear force to tear the tissue, then tissue cutting is not achieved. The inconsistent operation of the Tm only laser ablation makes practical application problematic.

Exemplary embodiments of the present disclosure address this shortcoming by providing an additional pulse at a laser wavelength that is strongly absorbed in the tissue (e.g. Er:YAG at 2.94 microns or CO₂ at 10.6 microns). The additional pulse at a laser wavelength that is strongly absorbed is absorbed in the most superficial layer in the tissue and effectively provides additional shear force so that the expanding/collapsing vapor bubble can tear the tissue and clean ablation can be achieved. The inventors have combined Tm (1.94 μm) and Er.YAG (2.94 μm) radiation. The apparatus demonstrated rapid and precise tissue ablation that is consistent and repeatable.

The application of two laser wavelengths achieved repeatable and consistent laser cutting of tissues. By applying the appropriate laser dosimetry for the two wavelengths, reliable and repeatable cutting can be achieved.

Exemplary embodiments accordingly provide an approach that allows the rapid and precise tissue cutting using currently available laser systems. While exemplary embodiments may have increased costs over some existing single laser systems, it is believed such costs will be more than offset by the increased efficiency in tissue removal rates while maintaining desired accuracy.

Exemplary embodiments provide an approach that allows the simultaneous (or near simultaneous) rapid and precise tissue cutting using relatively standard laser systems. For example, using femtosecond lasers, very precise tissue cutting can be achieved, however, the tissue removal rate is quite slow. The advantages of using a higher energy laser (including for example a Thulium (Tm) laser) for rapid tissue removal have also not been realized.

Laboratory experiments suggest that a problem associated with Tm only laser ablation is that the cutting is inconsistent and can result in a thermal runaway effect that can result in substantial tissue injury. Previous attempts at Tm only systems have not been successful due to these adverse effects. Exemplary embodiments of the present disclosure can address the issues encountered by Tm only laser systems by also applying a second laser pulse energy that is strongly absorbed. In one particular embodiment, the second laser has a wavelength was 2.94 μm emitted from an Er.YAG laser.

Certain embodiments include a method for subtractive material processing, where the method comprises a defect-inducing step and a bubble-generation step. In exemplary embodiments, the defect-inducing step directs radiation from an environment onto a material to create a spatially confined region with reduced mechanical modulus in the material between a bubble-generation site and an interface between the environment and the material; and the bubble-generation step directs pulsed radiation from the environment onto the material to create a subsurface bubble below the environment-material interface. In particular embodiments, material failure due to bubble expansion occurs and is enhanced by the material region with reduced mechanical modulus created by the defect-inducing step and results in material ejection.

In some embodiments, the material is cooled before, during or after the bubble generation step. In specific embodiments, the cooling is convective cooling. In certain embodiments, the cooling is evaporative cooling. In certain embodiments, the material is a biological tissue. In particular embodiments, the biological tissue contains a structural inhomogeneity. In some embodiments, the structural inhomogeneity is an epithelial tissue layer. In specific embodiments, the bubble-generation step creates a plasma. In certain embodiments, radiation emitted by an ultrafast laser creates the bubble in a material. In particular embodiments, the region of reduced mechanical modulus is conically shaped with least modulus reduction along the cone axis. In some embodiments, radiation for the defect inducing step is derived from the radiation source for the bubble generating step. In specific embodiments, radiation for the defect-inducing step is derived from pump-radiation for the radiation source for the bubble-generating step. In certain embodiments, radiation for the defect-inducing step is derived from the radiation source for the bubble-generating step through a non-linear conversion process.

In particular embodiments, the defect-inducing step utilizes radiation between 0.8-2.3 um. In certain embodiments, the bubble-inducing step utilizes radiation between 0.4-2.3 um. In particular embodiments, the defect-inducing step utilizes a ytterbium (Yt) fiber laser.

In some embodiments, the defect-inducing step utilizes an erbium (Er):Glass laser. In specific embodiments, the bubble-generation step utilizes a thulium (Tm) laser. In certain embodiments, the bubble-generation step utilizes an holmium (Ho):YAG laser.

Particular embodiments include a method of ablating tissue, where the method comprises: directing a first pulse of energy at a first wavelength to a region of tissue, where a vapor bubble is formed in the region of tissue after the first pulse of energy is directed to the region of tissue; and directing a second pulse of energy at a second wavelength to the region of tissue, where the second pulse of energy is directed to the region of tissue after the bubble in tissue is formed; and the second pulse of energy breaks the mechanical integrity of tissue surrounding the vapor bubble.

In some embodiments, the first wavelength is emitted by a thulium laser. In specific embodiments, the second wavelength is emitted by an erbium laser. In certain embodiments, the second wavelength is approximately 2.94 μm. In particular embodiments, the first wavelength is emitted by a holmium laser. In some embodiments, the second wavelength is emitted by a carbon dioxide laser. Specific embodiments further comprise viewing the bubble via optical coherence tomography. In certain embodiments, directing the first pulse of energy and the second pulse of energy to the region of tissue comprises directing the first pulse of energy and the second pulse of energy through a photonic crystal fiber. In particular embodiments, directing the first pulse of energy and the second pulse of energy to the region of tissue comprises directing the first pulse of energy and the second pulse of energy through a germanium dioxide fiber.

Certain embodiments include an apparatus comprising: a first laser configured to direct a first pulse of energy at a first wavelength to a region of tissue; a second laser configured to direct a second pulse of energy at a second wavelength to the region of tissue; and a control system configured to control operation of the first laser and the second laser. In particular embodiments, the control system is configured to control the duration of the first pulse of energy such that a vapor bubble is formed in the region of tissue after the first pulse of energy is directed to the region of tissue; the control system is configured to control operation of the first laser and the second laser such that a delay period of time exists between the first pulse of energy and the second pulse of energy; and the control system is configured to control the duration of the second pulse of energy such that the second pulse of energy breaks the mechanical integrity of the vapor bubble.

In some embodiments, the first wavelength is emitted by a thulium laser. In specific embodiments, the second wavelength is emitted by an erbium laser. In certain embodiments, the second wavelength is approximately 2.94 μm. In particular embodiments, the first wavelength is emitted by a holmium laser. In some embodiments, the second wavelength is emitted by a carbon dioxide laser. Specific embodiments further comprise a conduit configured to direct the first pulse of energy and the second pulse of energy to the region of tissue. In certain embodiments, the conduit comprises a photonic crystal fiber. In particular embodiments, the conduit comprises a germanium dioxide fiber.

Certain embodiments include a method of ablating tissue, where the method comprises: directing energy from a ytterbium laser to provide pre-coagulation of blood vessels in a region of tissue to be resected; directing energy from the ytterbium laser to increase the temperature of the region of tissue to be resected prior to resecting the tissue; directing energy from a thulium laser to resect tissue from the region of tissue to be resected; and directing energy from the thulium laser to provide post-resection coagulation. Particular embodiments further comprise a delay period between directing energy from the ytterbium laser to provide pre-coagulation of blood vessels in the region of tissue to be resected and directing energy from the ytterbium laser to increase the temperature of the region of tissue to be resected.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.” The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

As used herein, the terms “cut” (and related terms such as “cutting”, etc.) and “break the mechanical integrity” (and related phrases such as “breaking the mechanical integrity”) are used to refer to a process of breaking the molecular bonds in tissue.

As used herein, the term “light source” is understood to include any source of electromagnetic radiation, including for example, a laser. It is also understood that a “first light source” and a “second light source” may originate from a single laser. For example, a laser configured for operating under a first set of parameters (e.g. wavelength, amplitude, continuous wave or continuous pulse mode) may be considered a “first light source”, while the same laser configured for operating under a second set of parameters may be considered a “second light source.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The invention may be better understood by reference to one of these drawings in combination with the detailed description of specific embodiments presented herein.

FIG. 1 shows a schematic of an apparatus according to an exemplary embodiment.

FIG. 2 shows a flowchart of a method according to an exemplary embodiment.

FIG. 3 shows an image of a bubble resulting from a pulse of energy from a first light source of the embodiment of FIG. 1.

FIG. 4 shows an image of tissue shown after a second pulse of energy from the second light source is directed to the tissue.

FIG. 5 shows an enface view of tissue with ablated regions shown as the darker regions in the tissue.

FIG. 6 shows an enface view of tissue with ablated regions shown as the darker regions in the tissue.

FIG. 7 shows a section view of tissue before ablation.

FIG. 8 shows the section view of the tissue of FIG. 7 after ablation according to exemplary embodiments of the present disclosure.

FIG. 9 shows an enface view of ablated holes formed in porcine skin according to exemplary embodiments of the present disclosure.

FIG. 10 shows an enface view of ablated holes formed in porcine skin according to exemplary embodiments of the present disclosure.

FIG. 11 shows a schematic of an OCT guided laser surgery apparatus with co-aligned ytterbium and thulium (Yt/Tm) beams according exemplary embodiments of the present disclosure.

FIG. 12 provides an overview of aspects utilized in a method where a Yt/Tm laser combination was utilized to provide pre-coagulation of blood vessels and pre-heating of tissue prior to resection/ablation, followed by post-resection/ablation coagulation.

FIG. 13 provides a schematic view of the Yt and Tm laser amplitude versus time in the various aspects of procedures according to the present disclosure.

FIG. 14 displays results of a first example of mouse brain surgery using the Yt/Tm laser application as described herein.

FIG. 15 displays results of a second example of mouse brain surgery using the Yt/Tm laser application as described herein.

FIG. 16 displays results of a third example of mouse brain surgery using the Yt/Tm laser application as described herein.

FIG. 17 shows (A) Sample layered material corresponding to skin tissue. Highlighting the penetration depths and likely location of bubble formation for various lasers in comparison with material (skin) layers and their thicknesses. B) Blow off process in a homogeneous material.

FIG. 18 shows bubble formation and fractures (A) in a layered structure in a case of successful removal (B) of material.

FIG. 19 shows bubble formation in a layered structure resulting in fractures travelling along native inhomogeneities in the material. A) Bubble formation and subsequent fractures. B) Resulting void formation when either; fractures do not reach the surface or fractures are unable to shear the material completely and produce a blow-off event.

FIG. 20 shows (A) OCT enface image of a gelatin phantom before Tm laser irradiation. The black line shows a highlighted region selected for Tm ablation and controlled by the OCT system. (B) OCT enface image after Tm laser irradiates homogeneous gelatin phantom where line-cuts (1 mm and 400 μm) were created. (C) Cross-sectional image of the white-dotted-line highlighted in (B). Scale bars are 200 μm.

FIG. 21 shows enface and cross-sectional images obtained from OCT imaging for before and after cutting up to a phantom blood vessel that was embedded in a homogeneous gelatin sample at an angle with respect to the surface. (A) Enface image of the blood vessel that was embedded at an angle with respect to the surface of the tissue phantom. (B) Enface image after the laser cut. (C and D) Cross sectional images of the vessel before and after cutting. White arrows indicate the location of the vessel in the enface and cross-section images. Scale bars are 200 μm.

FIG. 22 shows (I) Illustration of vapor bubble formation and subsequent uncertainty that the bubble-generated stress is able to overcome the mechanical properties of tissue to eject the material and give a blow-off event. The browning highlighted in the bottom-left image is an indication of charring observed in cases where blow-off does not occur. (e.g. porcine skin samples). (II) Inconsistent ablation in the same tissue sample (uniformly cut pieces obtained from porcine-skin) at essentially adjacent locations. In (A) the bubble-generated shear stress is unable to induce sufficient microcracking and fracture to the material surface to eject material, whereas in (B) microcracking, and fracture to the surface occur and result in a material blow-off event. Scale bar is 250 μm.

FIG. 23 illustrates strategies to overcome the inconsistent blow-off from Tm laser bubble-generation alone. The method involves modification of tissue mechanical properties with a defect-induction step before/during nanosecond Tm laser bubble-generation pulse to remove material. Gradient in the visco-elastic modulus is achieved (B) with application of the defect-induction pulse that enhances micro-cracking and directs fractures to the surface in response to the bubble-generating pulse aiding in material failure at the surface resulting in a blow-off event (C).

FIG. 24 shows tissue removal rate for defect-inducing (Yt, 1.07 um, 120 um spot size) step followed by bubble-generation step (Tm laser, 1.94 um, 60 um spot size) in cartilage as a function of temperature increase and corresponding modulus variation. For pulse durations used, the Arrhenius integral threshold at 0>1 (red arrow) (from Diaz et.al. [1]) and Ω>0.1 (orange arrow). These were computed assuming a conservative estimate that temperature at the irradiation spot remains constant up to one thermal relaxation time.

FIG. 25 shows tissue removal rate for defect-inducing (Yt, 1.07 um, 120 um spot size) step followed by bubble-generation step (Tm laser, 1.94 um, 60 um spot size) in porcine skin as a function of temperature corresponding shear modulus variation. For pulse durations used, the Arrhenius integral (from Diller et.al. [2]) threshold of 0=1 (red arrow) and 0.1 (orange arrow). These were computed assuming a conservative estimate that temperature at the irradiation spot remains constant up to one thermal relaxation time.

FIG. 26 shows subtractive laser processing using defect-inducing (Yt, 1.07 um, 120 um spot size) step followed by bubble-generation step (Tm laser, 1.94 um, 60 um spot size) in porcine-skin imaged using OCT. (A) Enface images before and after laser irradiation employing Yt-laser (1.07 um, 120 um spot size) defect-inducing step followed by bubble-generation (Tm laser, 1.94 um, 60 um spot size). (B) Cross-sectional images of tissue removal in another ex vivo porcine-skin tissue specimen. (C) Multiple irradiations in a grid pattern at multiple locations.

FIG. 27 shows (A) Pre-surgery angiography of a control mouse (#C1); (B) Post surgery angiography of the control mouse (#C1). (C) Post surgery B-scan of cut produced by a Yt-laser (1.07 um, 120 um spot size) defect induction followed by Tm-laser (1.94 um, 60 um spot size) bubble generation. Cut required sequential application of about 5 such defect-inducing and bubble-generation steps; and (D) Corresponding histology of B-scan matching OCT measurements.

FIG. 28 shows a post-surgery OCT B-scan (left) of cut produced by a Yt-laser (1.07 um, 120 um spot size) defect induction followed by Tm-laser (1.94 um, 60 um spot size) bubble generation in an adjacent location to FIG. 27 cross-section. Cut required sequential application of about 5 (see FIG. 27) defect-inducing and bubble-generation steps; co-registered H&E stained tissue section (right) in an adjacent location to FIG. 27 (control mouse #C1).

FIG. 29 shows a biocompatible fiber (200 um core size) implementation for subtractive tissue processing with defect-inducing (Yt, 1.07 um) and bubble-generation (Tm, 1.94 um) radiation coupled into a single fiber for in vivo surgery.

FIG. 30 shows an in vivo surgery with biocompatible glass fiber implementation from FIG. 29. A) Pre-surgical angiography image; B) Post-surgery angiography image; C) B-scan intensity image at highlighted location of surgery using the biocompatible glass fiber.

FIG. 31 shows an illustration of strategies to achieve spatial patterning procedure (Step A) to extract material. The method involves modification of tissue with a conditioning pulse before/during short pulsed laser irradiation to create a bubble (Step B). Modulus gradient is achieved in an axicon shape (B) with the conditioning pulse that channels the fractures aiding the failure of the material along the axicon defect induction channels resulting in a blow off event (green arrow) (C).

### DETAILED DESCRIPTION OF ILLUSTRATIVE EMBODIMENTS

Exemplary embodiments of the present disclosure include apparatus and methods that provide efficient and precise ablation of tissues. It is understood that the embodiments described herein are merely exemplary, and that other embodiments are included within the scope of the invention.

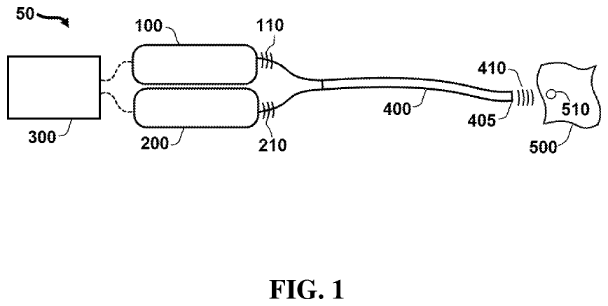

Referring now to FIG. 1, one exemplary embodiment of a system 50 comprises a first light source 100, a second light source 200 and a control system 300 configured to control operation of first and second light sources 200 and 300. In the embodiment shown, first light source 100 can be configured as laser that directs a first pulse of energy 110 at a first wavelength to a region of tissue 500. In addition, second light source 200 can be configured as laser that directs a second pulse of energy 210 at a second wavelength to region of tissue 500 in this embodiment. In particular embodiments, system 50 may comprise a conduit 400 configured to transmit first and second pulses of energy 110 and 210 from first and second light source 100 and 200 to region of tissue 500. Energy 410 (e.g. first pulse of energy 110 and second pulse of energy 210) from light sources 100 and 200 can then be directed to, and incident upon, region of tissue 500.

In certain embodiments, conduit 400 may be configured as a catheter with a distal end 405 that can be placed in proximity to region of tissue 500. In some embodiments conduit 400 may be comprise a photonic crystal fiber, and in specific embodiments conduit 400 may comprise a germanium dioxide fiber. In particular embodiments, the fiber or fibers can be housed in an extrusion and sealed so that risk of tissue contact can be appropriately mitigated.

During operation of the embodiment shown in FIG. 1, control system 300 is configured to control the duration of first pulse of energy 110 from light source 100 and second pulse of energy 210 from light source 200. In addition, control system 300 is configured to control operation of the first light source 100 and second light source 200 such that a delay period of time exists between first pulse of energy 100 and second pulse of energy 210.

In particular embodiments, control system 300 is configured to control the duration of first pulse of energy 110 such that a vapor bubble 500 is formed in region of tissue 500 after first pulse of energy 110 is directed to region of tissue 500. Control system 300 is also configured to control the duration of second pulse of energy 210 such that it reduces or breaks the mechanical integrity of tissue overlying the vapor bubble 500.

The ability to form a vapor bubble with a first energy pulse at one wavelength and reduce or break the mechanical integrity of the tissue overlying the vapor bubble with a second energy pulse at a second wavelength provides significant advantages over existing systems. For example, tissue ablation can be more precisely controlled and more efficiently performed than single wavelength systems. In particular, single wavelength system utilizing femtosecond lasers can provide precise tissue removal, but at a very slow rate. This can lead to extensive time required for procedures requiring significant volume of tissue ablation. Conversely, single wavelength system utilizing Thulium lasers can provide more rapid tissue removal, but without precise control. This can result in damage to healthy tissue that is not intended for removal or ablation. Exemplary embodiments of the present disclosure provide the ability to precisely and efficiently ablate tissue by utilizing a first wavelength to form a vapor bubble in the tissue and a second wavelength to reduce and/or break the mechanical integrity of the tissue overlying the vapor bubble (e.g. by ablating the layer of tissue covering the vapor bubble).

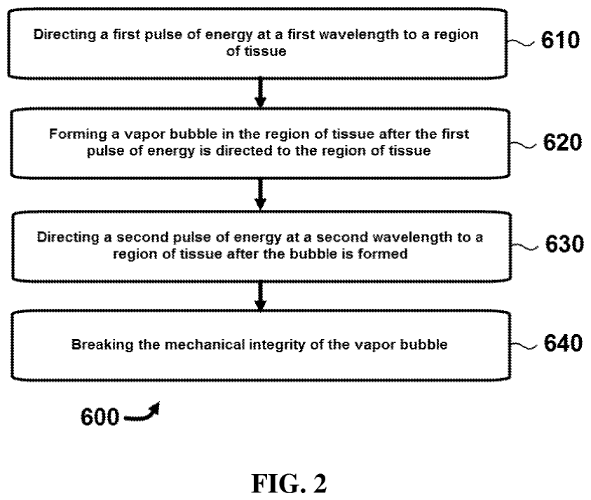

Referring now to FIG. 2, a flowchart 600 includes an overview of steps in a method of using an apparatus according to an exemplary embodiment, including for example, apparatus 50 shown in FIG. 1. Flowchart 600 comprises a first step 610 comprising directing a first pulse of energy at a first wavelength to a region of tissue, followed by a second step 620 of forming a vapor bubble in the region of tissue after the first pulse of energy is directed to the region of tissue. Next, flowchart 600 comprises a step 630 of directing a second pulse of energy at a second wavelength to a region of tissue after the bubble is formed. Finally, step 640 comprises reducing and/or breaking the mechanical integrity of the vapor bubble.

It is understood flowchart 600 provides only a general overview of steps in exemplary methods. Additional steps may be included in certain embodiments including, for example, viewing the bubble via optical coherence tomography.

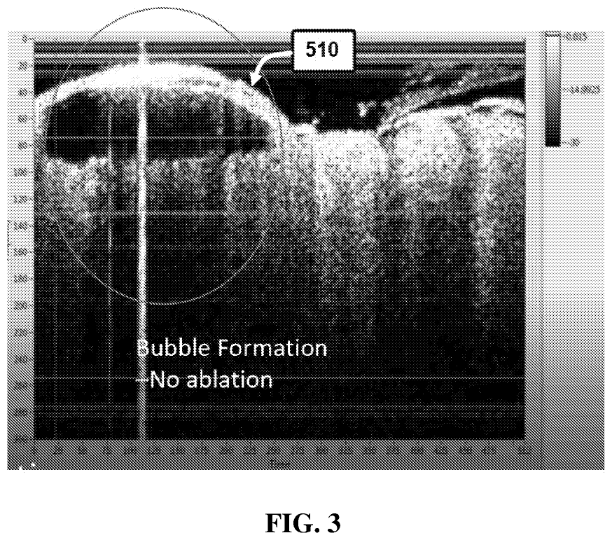

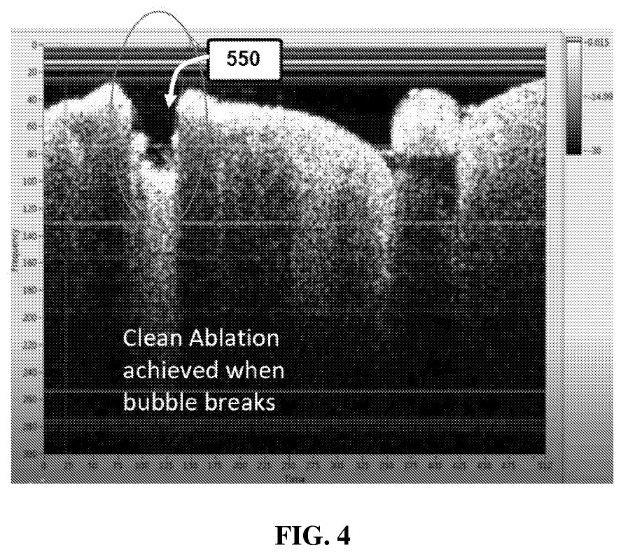

Referring next to FIG. 3, an image of bubble 500 is shown resulting from a pulse of energy from the first light source. FIG. 4 illustrates the results after the second pulse of energy from the second light source is directed to the same region of tissue shown in FIG. 3. As shown FIG. 4, an ablated region 550 results when the mechanical integrity of tissue overlying the bubble 500 has been reduced or broken.

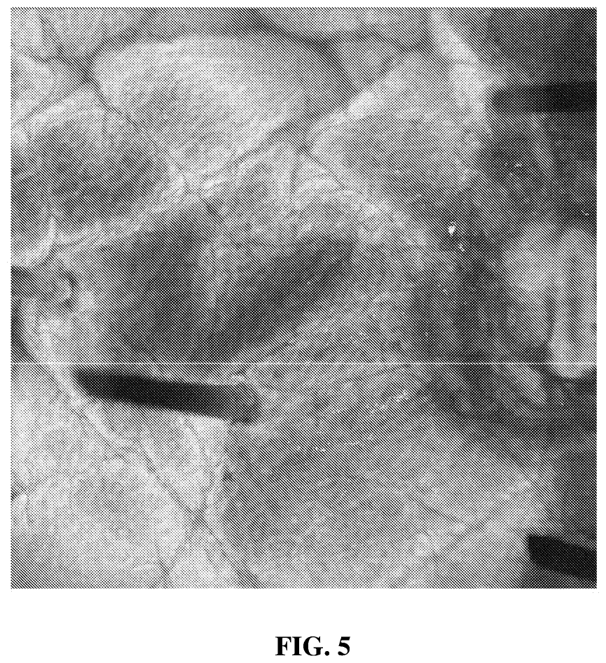

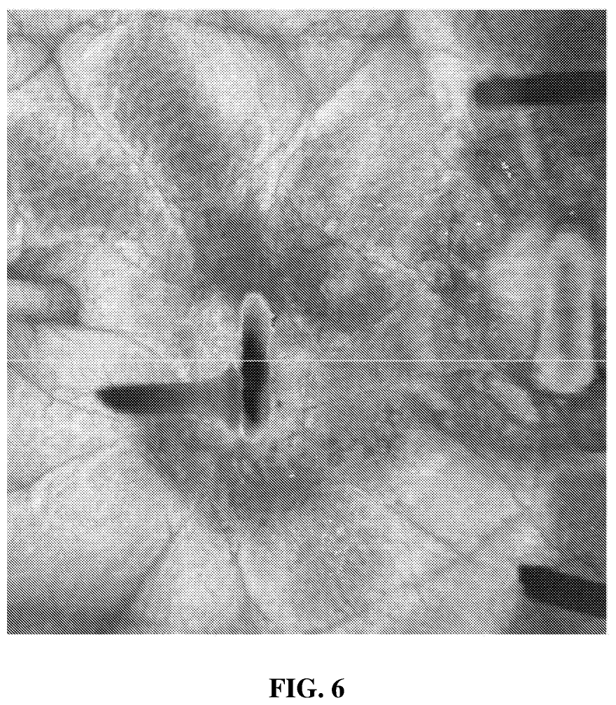

FIGS. 5 and 6 provide images of an enface view of tissue with ablated regions shown as the darker regions in the tissue. As shown in the figures, the ablated regions are well defined and can be precisely controlled.

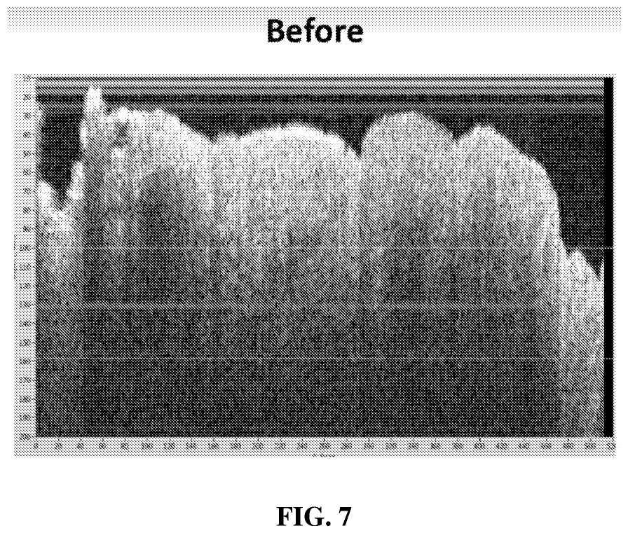

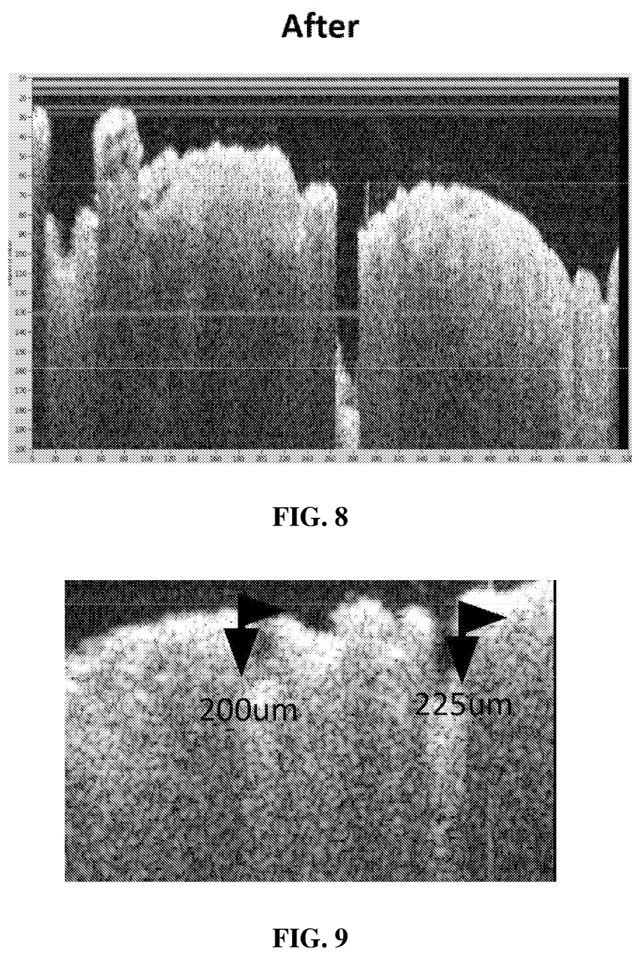

FIGS. 7 and 8 provide section views of tissue before and after tissue ablation is performed according to the present disclosure. Again the ablated region shown in the cross-section view is well defined.

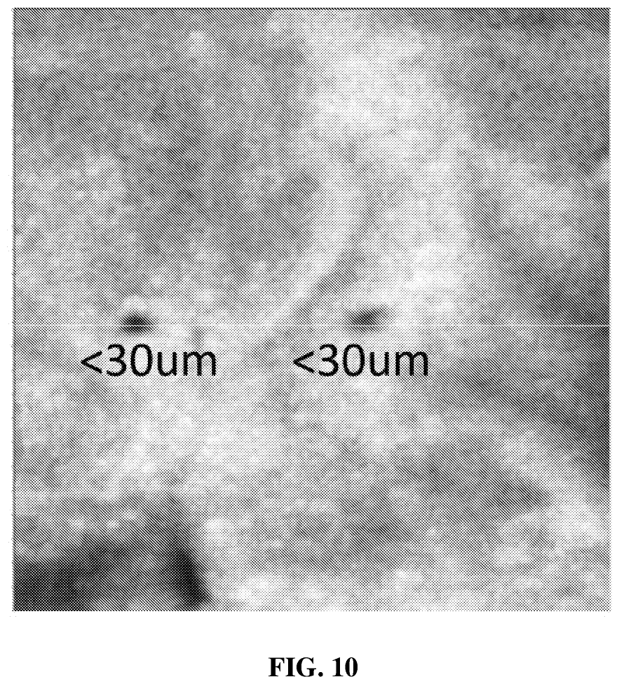

FIGS. 9 and 10 provide enface views of ablated holes formed in porcine skin using apparatus and methods as disclosed herein. As shown in the figures, the holes are less than 30 μm in diameter and approximately 200 and 225 μm deep.

Further description and explanation of the operating principles can also be found in the discussion of the examples and results that follow.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

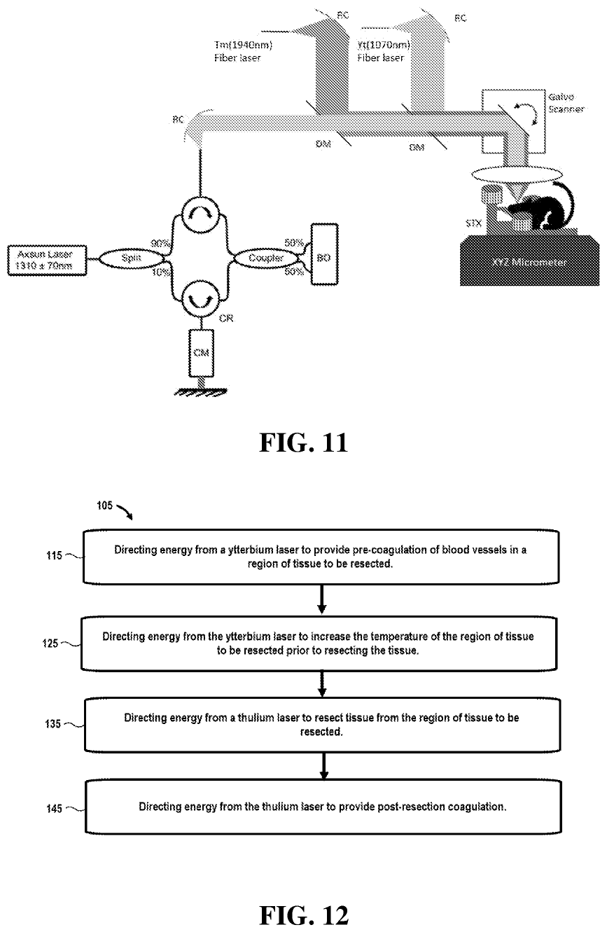

Referring now to FIG. 11, an OCT guided laser surgery apparatus is shown with co-aligned ytterbium and thulium (Yt/Tm) beams. It is understood that in this example, a Mach-Zehnder fiber Interferometer uses circulators (CR) and balanced detection (BD) and is dispersion compensated (CM). Tm/Yt and OCT beams are fiber delivered via collimators (RC) and combined with di-chroic mirrors (DM).

Utilizing the apparatus shown in FIG. 11, experiments were performed to demonstrate the effectiveness of methods disclosed herein. In particular, three brain surgeries were performed in control mice using a ytterbium fiber laser (1070 nm) for blood specific coagulation since the absorption in blood is differentially higher than surrounding native tissue. In addition, a thulium nanosecond fiber laser was used for removing brain tissue due to its higher tissue absorption.

In general, the Yt/Tm laser combination was utilized to provide pre-coagulation of blood vessels and pre-heating of tissue prior to resection/ablation, followed by post-resection/ablation coagulation. An overview of the aspects utilized in the method is provided in FIG. 12. Method 105 comprises a first aspect 115 in which energy is directed from a Yt laser to provide pre-coagulation of blood vessels in the region to be resected in order to avoid bleeding once tissue is removed. This is followed by aspect 125, which includes pre-heating of the region by Yt laser (e.g. by directing energy from the ytterbium laser to increase the temperature of the region of tissue to be resected prior to resecting the tissue). The pre-heating is performed to aid the Tm nanosecond tissue resection. In the embodiment shown in FIG. 12, the procedure then provides tissue resection by the Tm laser in aspect 135. In certain embodiments, the Yt laser may continue to emit during the Tm resection to minimize or eliminate bleeding after resection. In addition, the procedure utilizes the Tm laser to provide post-resection coagulation to reduce or prevent bleeding after local tissue removal in aspect 145 of method 105.

In specific embodiments, the initial coagulation step with the Yt fiber laser can be optimized to coagulate the full distribution of blood vessel sizes. For example, the dosimetry can be adjusted to coagulate all the vessel sizes (e.g. veins and arterioles) in the target area. The pre-heating aspect of the procedure (as a conditioning step to modify the shear modulus and viscosity of the tissue) using the Yt laser is a significant aspect of exemplary methods. In this aspect, the Yt laser can be used to transiently heat the target tissue up to a point where protein denaturation (outside the vasculature) is just about to occur (which will depend on an Arrhenius rate process for tissue surrounding the vessels). If the proteins surrounding the vessels denature during the pre-heating step, then the efficiency of the Tm resection decreases.

Accordingly, the Tm laser does the resection more efficiently with the pre-heating aspect disclosed herein.

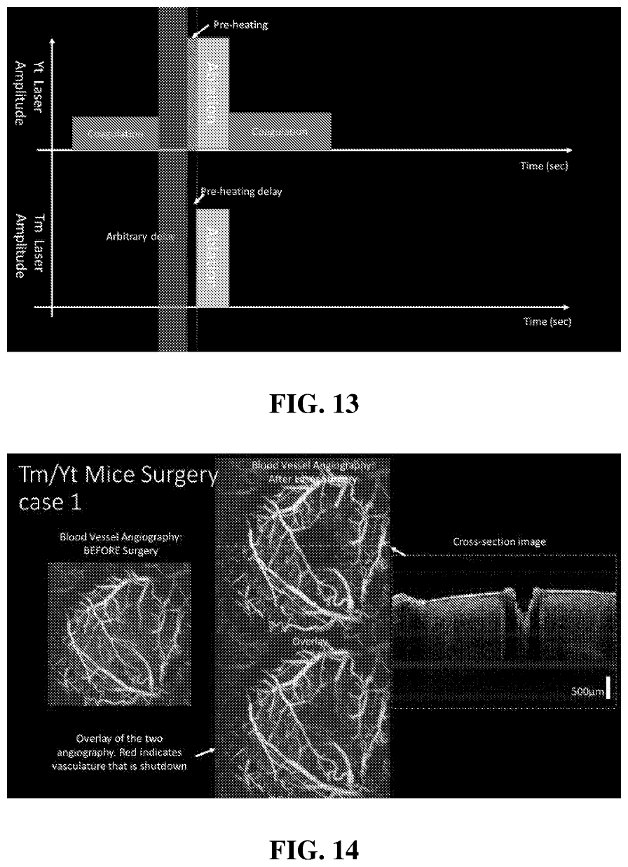

FIG. 13 provides a schematic view of the Yt and Tm laser amplitude versus time in the various aspects of procedures according to the present disclosure. In particular, FIG. 13 illustrates the Yt and Tm laser application during a pre-resection/ablation coagulation period, a delay period, an ablation period, and post-resection/ablation period as indicated in the figure.

FIG. 14 displays results of the first example of the mouse brain surgery using the Yt/Tm laser application as described herein. The image on the left shows blood vessel angiography before surgery. The image in the center top shows a blood vessel angiography after surgery, while the image in the center bottom shows an overlay of the images. The central (red) portion indicates the vasculature that has been shut down (e.g. resected/ablated). The image on the right is a cross-section of the tissue after surgery.

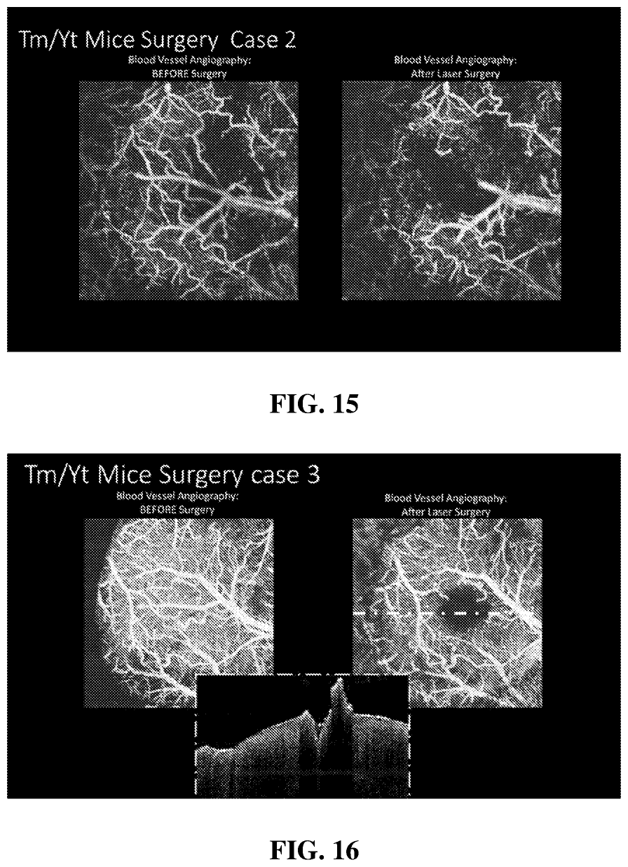

FIG. 15 displays the results of the second example of the mouse brain surgery using the Yt/Tm laser application as described herein. The image on the left is a blood vessel angiography before surgery, while the image on the right is a blood vessel angiography after surgery.

FIG. 16 displays the results of the third example of the mouse brain surgery using the Yt/Tm laser application as described herein. The image on the left is a blood vessel angiography before surgery, while the image on the right is a blood vessel angiography after surgery. The image at the lower center portion of the figure is a cross section of the tissue after surgery.

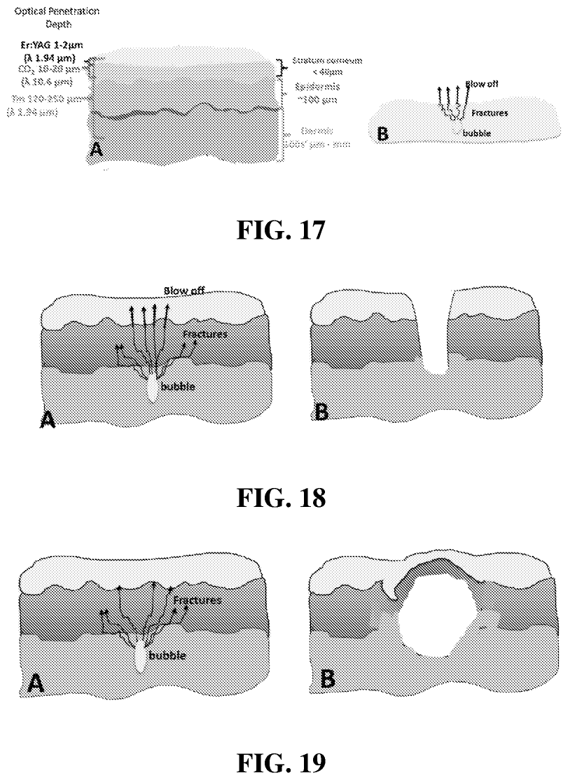

Referring now to FIG. 17, with laser-induced bubble creation and increased pressure due to gas and/or plasma creation, rapid bubble expansion occurs. In an elastic material that can support some shear stress (i.e., not a pure liquid), the free energy stored in high temperature gas and/or plasma contained within the bubble is converted into stored elastic energy in the surrounding material as the bubble expands. During rapid expansion and/or collapse of the laser-created bubble, existing material inhomogeneities are candidate sites that can fail and grow and enlarge into microcracks. Many types of material inhomogeneities are recognized in various materials (e.g. Skin, Panel A FIG. 17). Example material inhomogeneities can include but are not limited to, for example, structural interfaces, atomic or molecular interfaces, phase interfaces such as liquid/gas, protein/liquid, protein/gas, density gradients, and entropy defects or gradients. If laser-induced stresses are of sufficient magnitude, micro-crack growth is initiated at the material inhomogeneities and can cascade and become a larger scale fracture.

Cascading microcracks that become fractures will generally initiate along material inhomogeneity boundaries and propagate through the material. If the fracture propagates up toward the environment/material interface (i.e., air-material or liquid-material) and separates or parts the environmental/material interface and material overlying the bubble has sufficient momentum, material near the fractured region can “blow off” and result in a “cut” in the material which is a design objective of subtractive laser material processing (Panel B, FIGS. 17-18). Hence, the process of laser subtractive processing consists of bringing laser radiation incident on a material from the environment, realizing a sufficient fluence and/or fluence rate within the material to create a bubble that rapidly expands within the material, causing one or more material inhomogeneities to grow into micro-cracks due to laser-induced stresses.

Bubble-induced micro-crack growth develops and cascades into material fractures that can have a component of propagation normal to the environment-material interface, and finally result in a blow-off event with some of the material surrounding the bubble ejected into the environment (FIG. 18). In some cases, micro-crack growth is insufficient to result in fractures or fractures do not propagate to the material/environmental interface and a material blow-off event does not occur (FIG. 19). In other materials, as is frequently the case in biological tissues, a pre-existing wide-spread material inhomogeneity exists. When bubble creation is at or below the structural layer, fracture propagation can proceed along the lower structural boundary and not propagate up to the environment/material interface, so that a blow-off event does not occur (Panel B, FIG. 19). In cases when a blow-off event does not occur, laser subtractive processing fails and properties of the target material may be adversely affected.

For more efficient laser subtractive processing and higher material removal rates, bubble formation deeper in the material is required so that a greater volume of material overlying the bubble may be blown off giving more material removed per incident laser pulse. When bubbles can be created at deeper positions in the material, however, micro-crack growth resulting from bubble expansion becomes more unpredictable. For bubble formation at deeper positions, bubble expansion must be sufficient to create microcracks and eventually fractures that propagate to some degree along the interface normal and up to the environment-material interface and result in a material blow-off event. For deeper bubble formation that results in a material blow-off event, the pulse fluence or pulse fluence-rate required to generate an expanding bubble must be considered. For example, in cases when the pulse fluence or pulse fluence-rate is too low, although an expanding bubble might be created at a deeper position in the material, microcrack growth may be insufficient to create fractures that propagate to the environment-material interface and a blow-off event does not occur. Moreover, although increased pulse energy can be applied to create a more energetic bubble, existing material inhomogeneities may redirect microcracks and fractures either parallel or away from the environment/material interface so that a blow-off event does not occur.

In cases when fractures do not propagate up to and part or break the material/environment interface, at least three events can detract from the laser subtractive process. First, since material does not blow-off the intended material subtraction event does not occur. Second, thermal energy generated from the subsurface laser-material interaction becomes trapped inside the material and can result in localized non-specific melting, molecular unfolding, bond breaking and chemical modification. Third, resulting thermal damage, melting and molecular changes can modify the remaining material's functional, optical, mechanical, and chemical properties to not only prevent material subtraction with application of subsequent laser pulses but also have a deleterious effect on the material for the intended application.

In biological materials, although infrared laser radiation (0.8 um-2.6 um) can be used to create bubbles in tissue at positions deeper than 80 um below the environment-material interface, micro-crack growth and fracture propagation up to the environment-material interface may be unpredictable or insufficient to produce a blow-off event for subtractive processing. Some laser sources that target specific chromophores, like the water targeting Er:YAG (2.940 um) or CO₂ (10.6 um) lasers, allow for superficial bubble generation and precise tissue removal but are unable to achieve high aspect ratio cuts and do not provide high material removal rates. Conversely, although laser systems that provide deeper penetration of radiation in a target material do allow for bubble generation at deeper positions, micro-cracking and fracture generation is less predictable and has higher likelihood of not resulting in a material blow-off event. When a blow-off event does not occur, predictability of the action of subsequent laser pulses is sacrificed and material functionality may be lost. Non-specific thermal injury is especially problematic in some laser medical surgical procedures where, for example, non-specific thermal damage to neural tissues can result in patient disability.

Embodiments of this invention describe methods and various laser systems that allow for consistent and high material removal rates with high aspect ratios by creating a transient and spatially-confined viscoelastic inhomogeneity in the material so that when expanding bubbles are created at relatively deep positions, micro-crack growth and fracture propagation can reliably and repeatedly propagate to produce a blow-off event. The transient visco-elastic inhomogeneity created in the material may be designed so that microcracking and fracture generation due to bubble expansion propagates with a component along the normal to the environment-material interface to reliably and consistently result in a blow-off event with minimal residual non-specific damage to the remaining material. Accordingly, radiation sources, systems and methods are described that overcome existing limitations associated with laser subtractive systems and methods.

In the course of the development of a laser material removal system for application in biological tissues, the inventors constructed a thulium (Tm) laser system for rapid tissue cutting. Objective of the development effort was to leverage the deeper penetration of Tm laser light (compared to CO₂ or Er.YAG) in tissue (FIG. 17, Panel A) and realize higher volumetric tissue removal rates to meet the conflicting requirements of rapid and yet precise tissue removal for various laser surgical procedures. During development, the inventors observed serious limitations and unpredictability in using short-pulsed (100 ns) Tm lasers to cut or remove tissue. To investigate the material removal process, the inventors applied a Tm laser to numerous ex vivo and in vivo tissues and investigated the ablation process using optical coherence tomography (OCT) imaging ([3, 4]). The inventors observed that interaction of Tm radiation with many materials and tissues, bubble generation and material response was inconsistent and did not always provide for a blow-off event. OCT images recorded during Tm tissue irradiation, indicate tissue removal was inconsistent and that the Tm laser was unable to provide a clean and uniform ablation of both ex vivo and in vivo tissues including skin, muscle, brain, and adipose. Moreover, the inventors observed in many tissues non-specific thermal injury occurred with and without a blow-off event.

OCT imaging of the ablation process confirmed that when pulsed Tm laser light of sufficient fluence enters a target tissue, a rapidly expanding vapor bubble is formed. As described above, a laser material removal process involves shock wave propagation, bubble formation, expansion and collapse which results in large shear stresses in the material [5]. The stresses that accompany shockwave formation, bubble expansion and collapse interact with material inhomogeneities and if sufficient in magnitude result in microcrack growth and propagation of fractures. The observations in both ex-vivo and in-vivo tissues confirmed that although Tm laser irradiation with sufficient fluence could create a gas-filled bubble in tissue, the subsequent bubble expansion and collapse created unpredictable micro-cracking and fractures. In most cases, the fractures did not propagate up to the tissue interface and result in a blow-off event and material ejection. In some tissues such as skin, bubble-induced fractures propagated along an epithelial-tissue boundary and did not reach the air-tissue interface and result in a blow-off event and material ejection. If these cases, extensive non-specific residual thermal injury was observed in the tissue.

At this juncture, it is important to note observed differences in the interaction of pulsed Tm laser radiation with gelatin phantoms in comparison to various ex vivo and in vivo tissues. Experiments with gelatin phantoms highlight the effect that material inhomogeneities have in laser subtractive processing in actual tissues. Some differences between gelatin phantoms and tissue include: gelatin phantoms are a more homogeneous material with few native inhomogeneities in contrast with most tissues that have numerous types of inhomogeneities.

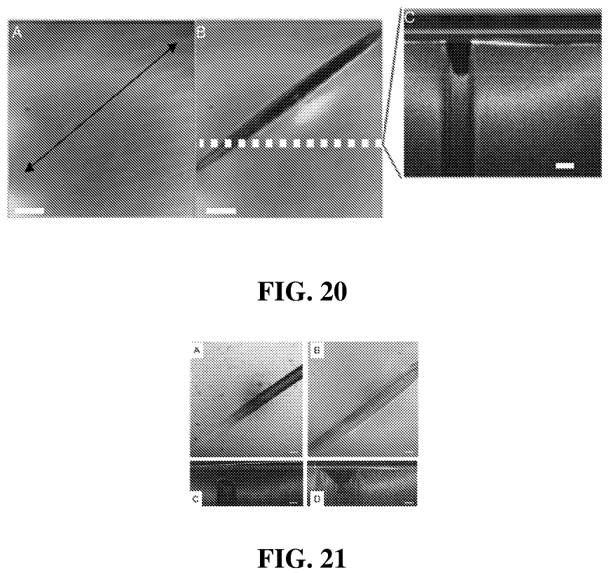

In many tissues, structural inhomogeneities exist that support function and can impact mechanical failure in response to bubble-creation. In gelatin phantoms, application of Tm pulsed radiation that did not consistently remove material in ex vivo and in vivo tissues, did consistently create a gas-filled bubble that resulted in a fracture propagating to the air-gelatin interface and give a blow-off event and material removal (FIGS. 20-21).

Tm laser ablation experiments with gelatin phantoms demonstrate the importance tissue inhomogeneities have in laser subtractive processing of heterogenous materials such as biological tissues. Although laser-induced bubbles can be generated in both gelatin phantoms and tissues, the subsequent microcracking and fracture propagation can be very different. In homogeneous gelatin phantoms, microcracking and fracture propagation to the surface with material blow-off was controlled and predictable. In contrast, with tissues although microcracking could be observed, fracture creation and propagation was highly unpredictable and frequently did not result in a material blow-off event. Moreover, in tissues increasing Tm pulse energy and/or fluence to create a more energetic bubble did not produce the desired effect of consistent and repeatable blow-off.

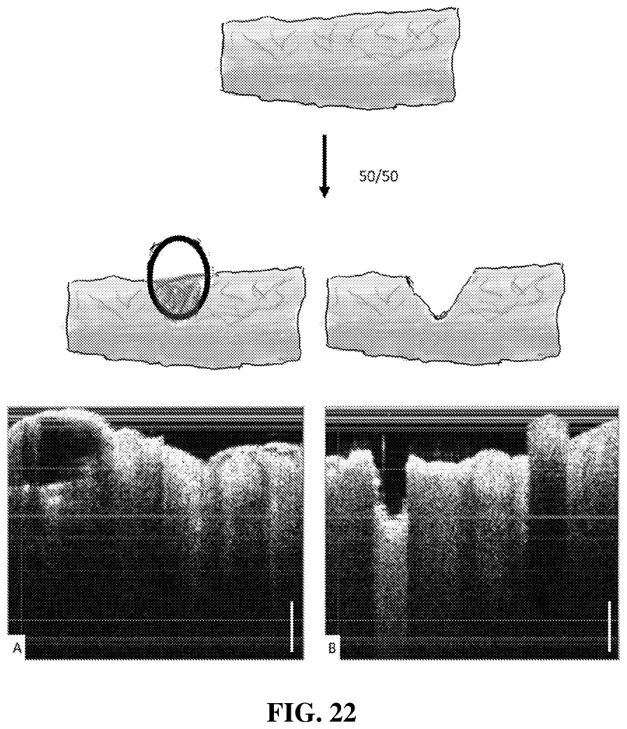

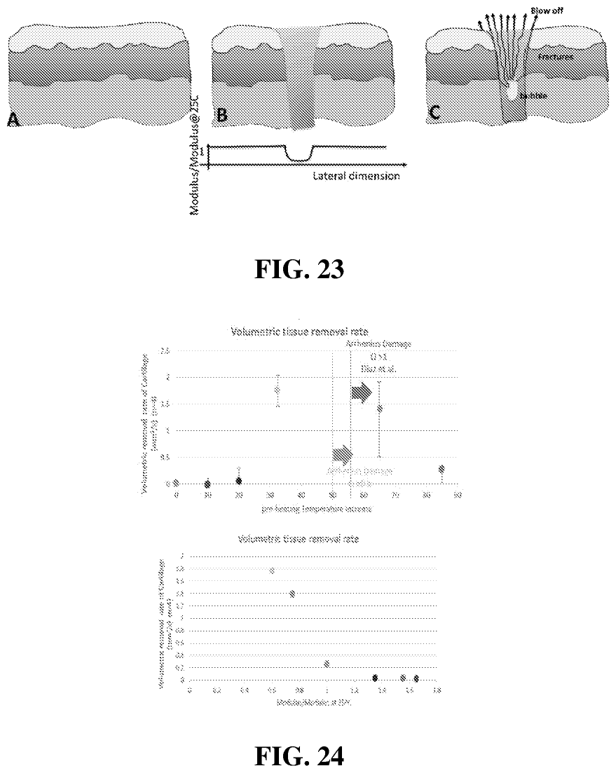

For example in tissue, even though the pulse duration and energy satisfy the entropy condition for water [6] and confinement resulting in little residual surface vaporization [6], as observable through OCT, collapse of the laser-created vapor bubble resulted in various scenarios. Two such scenarios are: 1) bubble-induced tensile and shear stresses sufficient to cause micro-cracking and tensile and/or shear failure with fracturing that propagates up to the tissue surface resulting in material ejection (FIG. 22, Panel I, IIB); or 2) bubble-induced tensile and shear stress sufficient to cause fracture formation that does not have a sufficient component along the interface normal so that material failure near the surface does not occur leaving hot debris trapped inside the tissue causing significant residual damage (FIG. 22, Panel IIA). Unfortunately, scenario 1 was inconsistent and unpredictable and could not be applied repeatedly for tissue cutting.

We describe a novel approach to aid laser subtractive processing by creating a transient visco-elastic inhomogeneity in the target material so that microcracking and fracture generation due to bubble expansion can propagate in a more predictable manner up to the environment-material interface to reliably and consistently result in a blow-off event. This innovative approach uses absorbed laser radiation (distinct from that utilized to create the bubble) to reduce the viscoelastic moduli of the target material to create a transient inhomogeneity that can then provide a measure of control over the preferred direction of micro-cracking, fracture generation and material blow-off in response to bubble generation. The approach is especially relevant when bubble generation is targeted at deeper positions in inhomogeneous materials since for those cases native material inhomogeneities can adversely prescribe the direction of preferred microcracking and fracture generation. In some cases, native material inhomogeneities might be unknown (even in a statistical sense) so that microcracking and fracture propagation in response to bubble generation propagates in a random manner making laser subtractive processing inconsistent and unreliable. In other cases, existing native material inhomogeneities may be well known and have a highly predictable orientation, however, microcracking and fracture generation along these existing boundaries can work in opposition to a bubble-induced blow-off event. In some biological tissues, for example, epithelial tissue boundaries can represent the primary material inhomogeneity and have a natural orientation parallel to the environmental/material interface. When laser-induced bubble generation occurs in these biological materials at a position just below or near an epithelial layer, resulting micro-cracking and fracture propagation has a higher likelihood of proceeding along the epithelial-tissue boundary. In these cases, laser induced bubble generation can form a fracture along the epithelial tissue boundary and a blow-off event does not occur.

Pulsed laser radiation may be utilized to create a spatially-controlled transient visco-elastic inhomogeneity in the target material. In many materials, the viscoelastic moduli are temperature dependent and take-on a reduced magnitude with a temperature increase that may be induced by absorption of pulsed laser radiation ([7, 8]). By reducing the viscoelastic moduli of the material in a spatially confined region near the bubble generation site, a transient inhomogeneity is created within the material. In material regions where the temperature is increased and viscoelastic moduli reduced, the strain (Eq. 1, where G is shear modulus) rate that is created by the shock wave, and expanding and collapsing bubble is increased. In material regions with reduced viscoelastic moduli, higher strain rate and spatial gradients in the strain rate results in more extensive micro-cracking that can more easily cascade into material fractures that—by design—propagate with a component along the interface normal to the surface resulting in material ejection and a blow-off event. Hence, pulsed laser radiation may be utilized to create a spatially-controlled transient visco-elastic inhomogeneity in the target material that directs fracture propagation to the surface giving a blow-off event. This concept of space-time controlled reduction of the viscoelastic properties of a target material to increase the strain rate, microcrack generation and fracture propagation can be applied not only in various inhomogeneous materials such as biological tissues but also in other material removal procedures like polymer processing, material processing, and related industrial processes.

$$Shear\_Strain = \frac{Shear\_Stress}{G} \qquad Eq . 1$$

Although a spatially-controlled transient visco-elastic inhomogeneity may be created in a target material to control and advance fracture propagation in response to bubble creation, physical constraints to the methodology are recognized for effective implementation. In polymer or protein-based materials, although a transient increase in temperature can reduce viscoelastic moduli ([7, 8]), a prolonged temperature increase over longer time durations can result in protein denaturation that increases viscoelastic moduli. Thus, if the time-integrated temperature increase to reduce the viscoelastic moduli is excessive or too-long, reduced strain rate resulting from bubble expansion and collapse can limit microcrack formation. Thus, the methodology we describe involves two steps that are described, respectively, as a defect-induction step (Step A) and a bubble-formation step (Step B). The bubble-formation step (Step B) creates a rapidly expanding bubble at a subsurface location in the material. Temporal relationship between the defect-induction step (Step A) and the bubble-generation step (Step B) is an important consideration and is dependent on the time-dependent optical, mechanical, chemical and thermal properties of the target material. For purposes of this discussion, we consider the bubble-generation step (Step B) to begin at time t=0 and is associated with laser pulse duration, τ_{B}. The defect-induction step (Step A) is assumed to begin at time t_{A,1} and continue through time t_{A,2} where these times are referenced to beginning of the bubble-formation laser pulse. During the defect-induction step (Δt_{A}=t_{A,2}−t_{A,1}) one or more depositions of laser radiation may be incident on the target material to control the size and spatial extent of the induced viscoelastic defect. Fine tuning the temporal relationship between defect-inducing and bubble-generation steps allows for optimal efficacy of material removal.

In some applications, commercial constraints limit the selection of light sources to complete Steps A and B. In these cases, the radiation to complete Step A may be derived from the laser source used to complete Step B. For example, the radiation for the defect-induction step (Step A) might by derived from the pump light source for the laser used for bubble-generation (Step B). Contemporary laser sources frequently utilize extremely bright fiber lasers or laser diodes as pump-sources so that radiation for defect-induction might be derived from these. For example, an approach to generate radiation for the defect-induction step (Step B) might directly utilize the pump source for the bubble generation laser. Alternatively, the defect-induction step may utilize a non-linear conversion process to shift the wavelength of either pump-light and/or laser emission from the laser utilized for bubble-generation (Step B). For example, Raman fiber lasers may be utilized to generate laser radiation over a wide-wavelength range in the infrared spectrum and represent a candidate approach to achieve a wavelength shift for a source for the defect-induction step (Step A).

A laser generated bubble may be formed that contains either a gas [9][10], plasma [11][12] or combination thereof ([10]). The laser wavelength (λ_{B}) utilized in the bubble formation step may be selected by utilizing a linear and/or non-linear absorption process in the target material. For plasma bubble formation, the non-linear or multiphoton absorption properties of the material are considered. Choice of the bubble formation laser wavelength(s) (λ_{B}) is(are) also driven by availability of the light source. Readily available laser wavelengths that target a linear absorption process in materials containing water (e.g., tissue) are Thulium/Ho:YAG (1.94 um/2.01 um). Short pulsed (picosecond to femtosecond) lasers can be utilized that provide for bubble generation utilizing—at least in-part—a non-linear absorption process. A practical utility of a non-linear absorption process for bubble generation is that material phase change resulting from light absorption is highly spatially localized to a target region in the material. Laser dosimetry [spot size, pulse duration, incident fluence] for the bubble formation step (Step B) utilizing either linear or non-linear absorption processes in a target material is known in the art [12] and can be configured to realize a sufficient fluence (J/cm²) or fluence rate (W/cm²) at a subsurface depth (zo) in the material where bubble generation is targeted. The laser dosimetry for Step B should account for the material optical properties including scattering (μₛ), anisotropy (g), and absorption (μₐ). Bubble generation in inhomogeneous scattering tissues can be achieved at deeper locations by employing various optical clearing approaches to reduce the scattering strength of the material [12].

Objective of the defect-induction step (Step A) is to create a controlled inhomogeneity in the material so that increased micro-cracking and reliable fracture propagation to the material/environment interface occurs in response to the bubble-generation step (Step B). In one embodiment, the defect-induction step (Step A) transiently increases material temperature (ΔT_{A}) using a light absorption (μₐ) process in a target material region generally at depths (z) between locus of bubble generation (zₒ) and the material/environment interface (z=0). In this embodiment, selection of the laser wavelength (λ_{A}) and dosimetry [spot size, pulse duration, incident fluence] for the material conditioning step (Step A) must consider light fluence (Φ) in the material region surrounding locus of bubble creation (Step B) and material thermal properties including heat capacity (C) and mass density (ρ), according to:

$$Δ {T}_{A} = \frac{{μ}_{a} Φ}{ϱ C} \qquad Eq . 2$$

Laser-generated material temperature increase (ΔT_{A}) in Step A must be sufficient to reduce material viscoelastic moduli to increase strain rate and microcrack formation to insure fracture propagation up to the material/environmental interface. Simultaneously, the laser generated material temperature increase (ΔT_{A}) in Step A must be sufficiently short (relative to the time of bubble creation and expansion) so that significant material phase change (e.g., thermal denaturation) that might limit microcrack expansion and fracture propagation does not occur.

In biological tissues, too large of a temperature increase (ΔT_{A}) over too long of a time duration (Δt_{A}) will result in protein denaturation and can adversely modify the induced defect. The Arrhenius integral and associated damage parameter (Ω, Eq. 3) provides a measure of the level of protein denaturation that occurs during the defect-induction step. Excessive protein denaturation (Ω˜1) is associated with a shear modulus increase and is another temporal consideration that constrains choice for time duration (Δt_{A}) of the defect-induction step.

$$Ω ( {t}^{′} ) = {\int }_{0}^{t ′} {A}_{o} \cdot exp ( \frac{- {E}_{A} ( T )}{{k}_{B} T} ) \cdot dT \qquad Eq . 3 )$$

In the art of laser-tissue thermal interactions, Arrhenius activation energy (Eₐ) per molecule and reaction rate (Aₒ) and critical temperature for tissue damage varies based on tissue type [13, 14], magnitude and time duration of temperature increase (ΔT_{A}) and spatial distribution in the tissue. For example, in skin, a critical temperature for damage typically ranges from 50-60° C. whereas in muscle it varies from 60-70° C. Temporally, this critical temperature for damage increases by 5° C. for every decade reduction in pulse duration. This suggests that use of a short-pulsed laser to reduce the defect-induction time duration (Δt_{A}) is desirable given the ability to heat tissue to higher temperatures before any (Arrhenius damage integral values indicate) tissue damage. By reducing defect-induction time duration (Δt_{A}) by utilizing a short-pulsed laser, higher temperatures may be achieved in some materials with greater reductions is viscoelastic moduli and consequent greater control over fracture propagation. Since Arrhenius damage is associated with increase in the shear modulus, designing conditioning time durations (Δt_{A}) short enough to reduce viscosity and bulk/shear moduli to increase strain rate and microcrack expansion while avoiding excessive molecular denaturation (Ω˜1) is desirable.

Another temporal consideration is defect-induction in the material during bubble expansion and collapse. In this case, t_{A,2} extends into the time of bubble expansion and/or collapse. Sequential strain rate impulses can enhance induction of defects and be achieved by delivering short duration laser pulses (e.g., ns) that are absorbed by material surrounding the bubble and rapidly decrease viscoelastic moduli and amplify the non-linear growth of microcracks and fracture propagation. Other temporal effects relate to sequential material blow-off events at one lateral position. To achieve sequential material blow-off events, the properties (e.g., temperature) of the material remaining from the previous blow-off event should be accounted for in the subsequent defect-inducting step. Thus, residual effects of the previous blow-off event, can cross-over to the subsequent defect-inducing step and reduce the required temperature increase (ΔT_{A}) of subsequent defect-inducing events.

Although the defect-induction step enhances each blow-off event and aids in removal of residual absorbed laser radiation, additional thermal energy may be removed from the material during and after the blow-off event by cooling. Non-specific residual thermal changes in the target material undergoing subtractive processing may be mitigated using a cooling approach. Cooling can be initiated prior to the bubble-generation step (Step B), during bubble expansion and collapse or after material blow-off. Various cooling approaches are recognized in the art including, for example, evaporation with phase change, convection and conduction. Approaches that utilize conduction must be configured so that the medium heat is conducting into (i.e., from the material undergoing subtractive laser processing) does not substantially interfere with neither blow-off events nor with subsequent defect-inducing events. Passive or forced gas convective cooling provides the advantages of enhanced convective removal of blown-off material together with not interfering with neither blow-off events nor subsequent defect-inducing events. Although evaporation with phase change normally has a higher heat-transfer coefficient compared with either conductive or convective cooling approaches, residence time of the phase-change material may lengthen application time of any required subsequent defect-inducing steps.

Spatial patterning (grid pattern irradiation) is possible for Steps A and B allowing for material removal at said grid-points. A plethora of spatial patterns can be envisioned where resection is made possible by controlling the spatial distribution of temperature increase of the defect-inducing step and application of the bubble-inducing step. By spatially patterning temperature increase of the defect-inducing step, microcrack expansion and fracture propagation can be spatially confined to selected regions in the target material. For example, utilizing axicons for the defect-inducing step can be configured to generate a surface-confined conical region ([15, 16]) so that fracture propagation and material blow-off is spatially controlled and limited to a conical region. The axicon configuration combined with bubble-generation using an ultra-short pulsed laser and multi-photon absorption and plasma generation can provide for material blow-off with minimal thermal modification to a relatively large tissue volume. This configuration can be useful for tissue harvesting or micro-biopsy so that a diagnostic screening approach can be applied characterize harvested tissue. Micro-biopsy with rapid screening of tissue with optical (e.g., Raman), mass-spectrometry or radio-frequency approaches can aid cancer surgeries.

The following considers the above factors in tissue resection and an example co-linear embodiment with a controlled temporal reduction of shear modulus tested in ex vivo and in vivo tissues for surgery.

The defect-induction step (Step A) was tested using a Ytterbium (Yt) fiber laser (1.07 μm) by applying a laser pulse co-aligned with radiation for the bubble-generating pulse to induce a space-time controlled inhomogeneity in the target material for subtractive laser processing. The bubble generation step (Step B) utilized a Thulium fiber laser (1.94 um wavelength) co-aligned with radiation (1.07 um) emitted by the Yt fiber laser for defect generation. At the environment/material interface, light emitted from the Yt fiber laser irradiated a slightly larger lateral region than that from the bubble-generating light source (in this example a Thulium fiber laser). Absorbed Yt laser light increases temperature in the target material and reduces the viscoelastic moduli of the material so that bubble-generation by the Tm laser reliably results in a material blow-off event. The approach is illustrated in FIG. 22, where the laser (Yt) generated transient defect is illustrated as a brown colored region. The material (in this case tissue) removal rate in this scheme is only limited by average power of the bubble-generating light source (in this case a thulium fiber laser which over the past few years has increased by orders of magnitude from a relatively small 15 W value (Ahmadi et al. 2017; Katta et al., 2017.) to 1 kW—an increase of 66.7×. Different temperature increases (ΔT_{A}) were induced in the defect-induction step in various tissue types (Table 1). Equation 2 above was applied to evaluate Yt laser dosimetry to achieve specified temperatures.

$$Δ {T}_{A} = \frac{{μ}_{a} Φ}{ϱ C}$$

Here, μₐ corresponds to the absorption coefficient at 1.07 um (about 0.1 cm⁻¹), fluence is computed for a spot size of 120 μm, ρ and C are density of tissue and specific heat (together their product yields about 0.004 J/mm³/K). Yt laser's pulse duration and peak power were adjusted to provide a ΔT_{A}=10, 20, 32.5, 65, 85 C computed temperature increase just before the bubble-generating laser radiation (Tm laser) entered the tissue. The limiting peak power of the Yt laser module was 3000 W (10% max. duty cycle, 300 W Avg.), the repetition rate and pulse duration were fixed at 50 us/100 us/125 us depending on the temperature increase requirement and repetition rate was adjusted to account for a 10% duty cycle limitation (2000 Hz, 1000 Hz and 800 Hz for 50/100/125 us respectively).

TABLE 1
Defect-induction computed temperature increases from Yt fiber
irradiation in porcine skin

| Pulse duration | Peak power | Approx. Temperature |
|---|---|---|
| (us) (Δt_{A}) | value (W) | Increase (° C.) ΔT_{A} |
| 50 | 1000 | 10 |
| 50 | 2000 | 20 |
| 50 | 3000 | 32.5 |
| 100 | 3000 | 65 |
| 125 | 3000 | 85 |

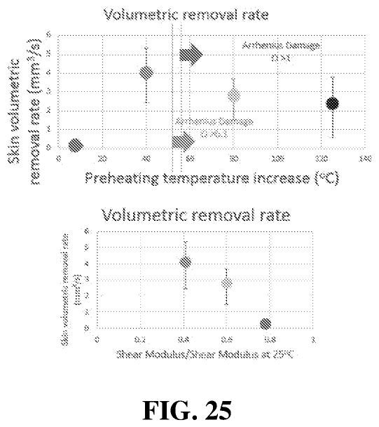

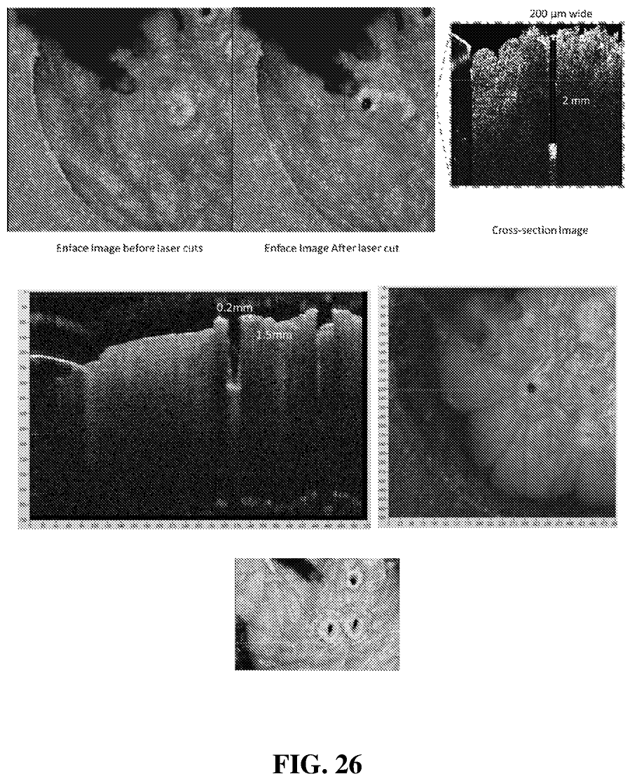

Porcine skin (n=2, 4 locations per sample) and porcine cartilage (n=2, 4 locations per sample) samples were utilized to test configuration of dual wavelength Yt/Tm lasers by first performing the defect induction step (Step A) with a Yt fiber laser (1.07 um), followed by bubble-generation (Step B) using a Tm laser (1.94 um, 300 uJ pulse energy, 100 ns pulse duration, 50 kHz repetition rate for 5 ms duration). Time delay between the defect-induction and bubble-generation steps was fixed so that Tm pulsed laser irradiation instantaneously followed the Yt laser defect induction step (i.e., t_{A2}=0). As mentioned in the methods section, Yt laser's pulse duration and peak power were adjusted to provide various temperature increases (ΔT_{A}, Table 1) before bubble-generating Tm laser radiation entered the tissue. FIGS. 24-25 show the volumetric tissue removal rate for Tm laser bubble-generation obtained for different ATA induced in the defect-induction step (Step A) resulting from Yt laser irradiation. Defect-induction temperature increases between ΔT_{A}=30-50° C. gave higher tissue removal rates for a fixed Tm bubble-generating pulse energy. Higher defect-induction temperature increases resulted in less effective tissue removal rates. Measured tissue Tm removal rates are close to the calculated values from modeling results [4] in these examples. Cartilage being mechanically stiffer (i.e., higher modulus) than skin yielded lower removal rates overall compared to skin, although possessing similar water content (% water content) (from simulation results of 70% water content tissues obtained from [4] for homogeneous gelatin phantoms). From previously reported studies of the temperature dependence of bulk moduli in cartilage (e.g., shear modulus in soft tissue among others) [7, 8, 18], the defect-inducing step (Step A) produces an inhomogeneous region of shear modulus reduction corresponding to temperatures in range of 60-70° C. thus enhancing microcracking and fracture propagation to the tissue surface and aiding material blow-off immediately following the Tm laser bubble-generating step (Step B). Conservative estimates of the Arrhenius integral (Ω) were computed assuming a constant temperature up until a thermal relaxation time and highlighted for two tissues in FIG. 24 (porcine cartilage) and FIG. 25 (porcine skin) (red arrow Ω>1 and orange arrow Ω>0.1). In actual practice, these estimates would be less conservative (threshold temperatures for Ω=0.1 and Ω=1 would be higher than those presented in FIGS. 24-25) due to thermal diffusion into the tissue surrounding the defect-inducing laser irradiation spot. High aspect ratio porcine skin tissue removal (FIG. 26) is made possible by sequential application of repeated defect-induction (Step A, Yt laser) followed by a bubble-generation (Step B, Tm laser) steps.

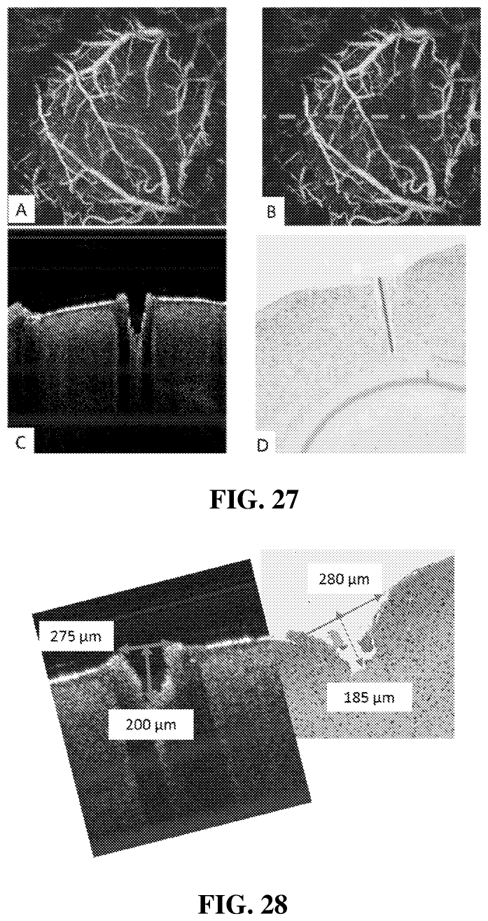

An image of the pre-operative vascular network (Panel A, FIG. 27) was overlaid on a post coagulation angiography image (Panel B) showing clear coagulation margins. Post-coagulation subtractive material processing using a Yt-laser defect-induction step (Step A) followed by Tm-laser bubble-generation (Step B) resulted in consistent material blow-off and cuts during surgery while the surgical field remained bloodless. Dimensions of the vertical channel created in tissue using the defect-induction and bubble-generation steps using OCT matched values determined from histology (FIGS. 27-28). The defect induction step (Step A) followed by the bubble-generation (Step B) allowed for creation of vertical channels with little observed thermal damage.

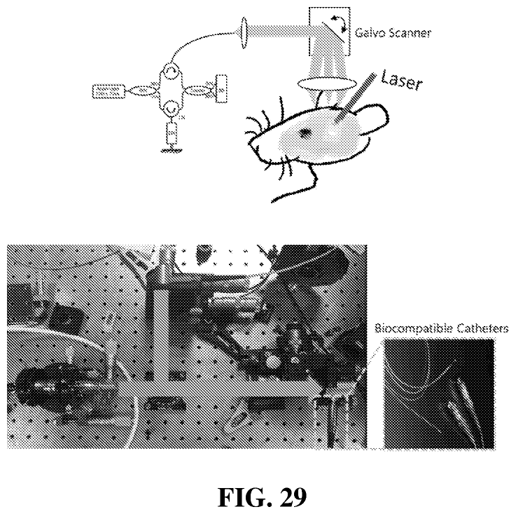

The methodology of a laser defect-induction step (Step A) followed by a bubble-generation step (Step B) was also tested for in vivo surgical applications using a fiber catheter by coupling both Yt (1.07 um) and Tm laser (1.94 um) radiation into a single multimode optical fiber. For Tm laser radiation, although fluence rate at the fiber tip was reduced by a factor of 10 compared to an open-air bulk-optics system, fluence values were slightly above threshold for bubble-generation. Tm light exiting a fiber with a core size of 10 μm coupled into the 200 μm core diameter fiber with reduced fluence rate at the fiber tip (with 80-90 percent coupling efficiency), while Yt light (50 μm fiber) coupled with a slightly lower efficiency of about 70 percent (FIG. 29, bottom).

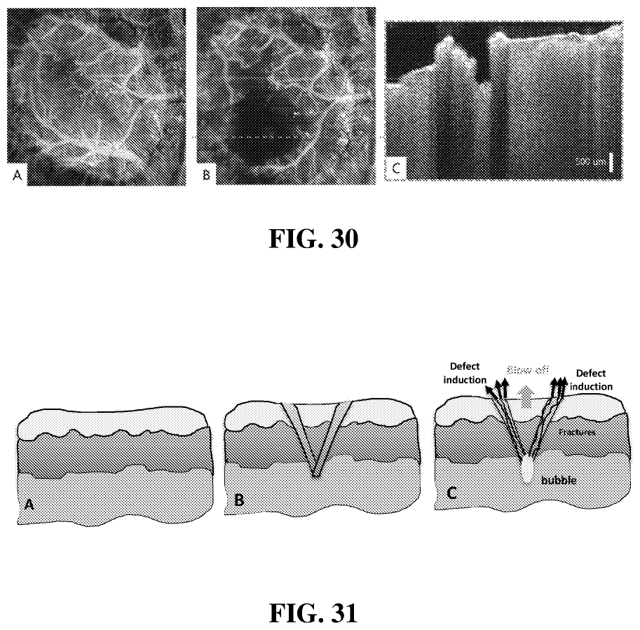

In vivo biocompatible glass fiber implementation for tissue subtractive processing using a 200 μm core fiber, resulting in a lower fluence rate for the bubble-generation step (Step B), provided material blow-off and successful tissue removal for surgery (FIG. 30). A post-surgery angiography image (Panel B, FIG. 30) shows a close-up view of Yt-laser (1.07 um) blood vessel coagulation compared to pre-surgery angiography (Panel A, FIG. 30). Removal of murine brain tissue (#C7) took longer than in an open-air system due to a lower bubble-generating fluence rate resulting in smaller tissue removal rates.

Co-linear propagation of defect-induction and bubble-generating beams has been successfully demonstrated and several other embodiments are recognized that might be derived from the results presented above. As mentioned previously, for tissue, choice of wavelength allows for multiple laser wavelength combinations for the defect-induction (Step A) and bubble-generation (Step B) steps. Some laser combinations for surgery in biological tissues include: Yt (Step A, 1.07 um)/Tm (Step B, 1.94 um), Yt (Step A, 1.07 um)/Ho (Step B, 2.06 um), Er:Glass (Step A, 1.55 um)/Tm (Step B, 1.94 um), Er:Glass (Step A, 1.55 um)/Ho (Step B, 2.06 um), Tm (Step A, 1.94 um)/Ho (Step B, 2.06 um), In-band Tm/Ho[19] (Step A in-band pumping at 1.9 um, Step B output laser pulse 2 um). One skilled in the art of laser tissue interactions may recognize other possible laser combinations. For these combinations, design of the temporal intensity and timing is completed in a manner analogous to Yt/Tm steps for both defect-induction and bubble-generation. Other combinations can be envisioned in the 2 um mid-infrared spectral regions in addition to these 2 um IR regions. Also, instead of using an IR laser (e.g., Tm or Ho or wavelength tuned Tm) for the bubble generation step—an embodiment uses a short-pulsed laser (picosecond to femtosecond) source that has very little linear absorption but has sufficient fluence for non-linear absorption near the focus to generate a plasma and bubble. This approach of using a non-linear bubble-generation step can better preserve the harvested tissue since the irreversible damage will be localized to the region where the bubble is created. Complementary to a bubble induction step that uses short-pulsed laser radiation (picosecond to femtosecond) is a defect-induction step that produces a spatially patterned temperature increase. Spatial-patterning of the defect-inducing step allows microcrack expansion and fracture propagation to be spatially confined to selected regions in the target material. For example, utilizing axicons for the defect-inducing step can be configured to generate a surface-confined conical region so that fracture propagation and material blow-off is spatially controlled and limited to a conical region (FIG. 31). The axicon configuration combined with bubble-generation using a short-pulsed laser and multi-photon absorption and plasma generation can provide for material blow-off with minimal thermal modification in a relatively large tissue volume. This configuration can be useful for tissue harvesting or micro-biopsy so that a diagnostic screening approach can be applied characterize harvested tissue.

Other spatial-patterning embodiments allow for an array (scanning or lenslet array) of laser irradiation sites with co-linear propagation of defect-inducing and bubble-generation beams where a relatively large bubble-generation pulse energy may be distributed over the environment/material interface together with defect induction steps. Spatial patterning of defect-induction laser radiation and depth distribution of viscoelastic moduli reduction (up to the time of ablation or even as the bubble expands and/or collapses) allows for microcrack generation and fracture propagation along controlled channels to accelerate resection along these channels of patterned irradiation. Spatial patterning (grid pattern irradiation) is possible for the defect-induction step allowing for material blow-off at only said grid-points while the entire region is irradiated by a bubble-generating light source (Panel C, FIG. 26 through scanning or lens-let array). A plethora of spatial patterning geometries can be envisioned where material blow-off is made possible by controlling the spatial-pattering of laser irradiation utilized in the defect-induction step.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
1. Díaz S H, Aguilar G, Basu R, Lavernia E, Wong B J F. Modeling the Thermal Response of Porcine Cartilage to Laser Irradiation. 2002; 4617: 47-56.
2. Diller K R, Klutke G A. Accuracy analysis of the Henriques model for predicting thermal burn injury. 1993; 268: 117-123 B T-Advances in Bioheat and Mass Transfe.
3. Katta N, Mcelroy A, Estrada A, Milner T E. Optical coherence tomography (OCT) guided smart laser knife for cancer surgery (Conference Presentation). Proc.SPIE 2017; 10054:
4. Katta N, McElroy A B, Estrada A D, Milner T E. Optical coherence tomography image-guided smart laser knife for surgery. Lasers Surg. Med. August 2017 Vol 50.
5. Wen S, Schmidt M S, Thomas R J, Wen S, Ly K, Bhaskar A, Schmidt M S, Thomas R J. Direct numerical simulation of the initial stage of a thermally induced microcavitation in a water-rich biotissue triggered by a nanosecond pulsed laser. J. Biomed. Opt. 2017; 22:
6. Welch A J, Van Gemert M J C. Optical-thermal response of laser-irradiated tissue. 2011;
7. Brosses E S, Gennisson J, Pernot M, Fink M, Tanter M. Temperature dependence of the shear modulus of soft. 2010; 1701:
8. Romodina M, Li J, Singh M. Measurement of the temperature dependence of Young's modulus of cartilage by phase-sensitive optical coherence elastography. 2014;
9. Wen S, Schmidt M S, Thomas R J, Wen S, Ly K, Bhaskar A, Schmidt M S, Thomas R J. Direct numerical simulation of the initial stage of a thermally induced microcavitation in a water-rich biotissue triggered by a nanosecond pulsed laser. J. Biomed. Opt. 2017; 22:
10. Vogel A, Venugopalan V. Mechanisms of Pulsed Laser Ablation of Biological Tissues. 2003;
11. Oraevsky A A, Silva L B Da, Rubenchik A M, Feit M D, Glinsky M E, Perry M D, Mammini B M, Small W, Stuart B C. Plasma mediated ablation of biological tissues with nanosecond-to-femtosecond laser pulses: relative role of linear and nonlinear absorption. IEEE J. Sel. Top. Quantum Electron. 1996; 2: 801-809.
12. Qiu J, Neev J, Wang T, Milner T E. Deep Subsurface cavities in skin utilizing mechanical optical clearing and femtosecond laser ablation. Lasers Surg. Med. 2013; 45: 383-390.
13. Welch A J, Pearce J A, Diller K R. RATE PROCESS MODELING OF LASER INDUCED THERMAL DAMAGE IN TISSUE.
14. Agah R, Pearce J A, Welch A J. Rate Process Model for Arterial Tissue Thermal Damage: Implications on Vessel. 1994; 184:
15. Inoue R, Takakusaki K, Takagi Y, Yagi T. Micro-ablation on silicon by femtosecond laser pulses focused with an axicon assisted with a lens. Appl. Surf. Sci. 2010; 257: 476-480.
16. Ren Q, Birngruber R. Axicon: A New Laser Beam Delivery System for Corneal Surgery.

IEEE J. Quantum Electron. 1990; 26: 2305-2308.
17. Ahmadi P, Estrada A, Katta N, Lim E, McElroy A, Milner T E, Mokan V, Underwood M. 1940 nm all-fiber Q-switched fiber laser. SPIE 2017; 10083: 100830G-10083-7.
18. Chae Y, Aguilar G, Lavernia E J, Wong B J F. Characterization of Temperature Dependent Mechanical Behavior of Cartilage. 2003; 278: 271-278.
19. Sincore A, Cook J, Shin D J, Roumayah P, Bodnar N, Richardson M. System design for a >1 kW in-band pumped thulium-doped fiber amplifier (Conference Presentation). Proc.SPIE 2019; 10897:
20. Watanabe et al.; Synchronous radiation with Er.YAG and Ho:YAG lasers for efficient ablation of hard tissues; 1 Sep. 2010/Vol. 1, No. 2/BIOMEDICAL OPTICS EXPRESS 337.
21. Schmidlin et al.; Enhancement of Tissue Lesion Depth by Dual Wavelength Irradiation with the Nd-YAG/KTP Laser: Perspectives for Laser Prostatectomy; Lasers in Medical Science 1997, 12:364-370.
22. ABSTRACT, Pratisto et al., Combination of fiber-guided pulsed erbium and holmium laser radiation for tissue ablation under water, Appl Opt. 1996 Jul. 1; 35(19):3328-37.

1. A method for subtractive material processing, the method comprising:
a defect-inducing step; and
a bubble-generation step, wherein:
the defect-inducing step directs radiation from an environment onto a material to create a spatially confined region with reduced mechanical modulus in the material between a bubble-generation site and an interface between the environment and the material;
the bubble-generation step directs pulsed radiation from the environment onto the material to create a subsurface bubble below the environment-material interface; and
material failure due to bubble expansion occurs and is enhanced by the material region with reduced mechanical modulus created by the defect-inducing step and results in material ejection.

2. The method according to claim 1 wherein the material is cooled before, during or after the bubble generation step.

3. The method according to claim 2 wherein the cooling is convective cooling.

4. The method according to claim 2 wherein the cooling is evaporative cooling.

5. The method according to claim 1 wherein the material is a biological tissue.

6. The method according to claim 5 wherein the biological tissue contains a structural inhomogeneity

7. The method according to claim 6 wherein the structural inhomogeneity is an epithelial tissue layer

8. The method according to claim 1 wherein the bubble-generation step creates a plasma

9. The method according to claim 8, wherein radiation emitted by an ultrafast laser creates the bubble in a material.

10. The method according to claim 1 wherein the region of reduced mechanical modulus is conically shaped with least modulus reduction along the cone axis.

11. The method of claim 1 wherein radiation for the defect inducing step is derived from the radiation source for the bubble generating step.

12. The method of claim 11, wherein radiation for the defect-inducing step is derived from pump-radiation for the radiation source for the bubble-generating step.

13. The method of claim 11, wherein radiation for the defect-inducing step is derived from the radiation source for the bubble-generating step through a non-linear conversion process.

14. The method of claim 5, wherein the defect-inducing step utilizes radiation between 0.8-2.3 um.

15. The method of claim 5, wherein the bubble-inducing step utilizes radiation between 0.4-2.3 um.

16. The method of claim 5, wherein the defect-inducing step utilizes a ytterbium (Yt) fiber laser.

17. The method of claim 5, wherein the defect-inducing step utilizes an erbium (Er):Glass laser.

18. The method of claim 5, wherein the bubble-generation step utilizes a thulium (Tm) laser.

19. The method of claim 5, wherein the bubble-generation step utilizes an holmium (Ho):YAG laser.

20. A method of ablating tissue, the method comprising:
directing a first pulse of energy at a first wavelength to a region of tissue, wherein a vapor bubble is formed in the region of tissue after the first pulse of energy is directed to the region of tissue; and
directing a second pulse of energy at a second wavelength to the region of tissue, wherein:
the second pulse of energy is directed to the region of tissue after the bubble in tissue is formed; and
the second pulse of energy breaks the mechanical integrity of tissue surrounding the vapor bubble.

21. The method of claim 20 wherein the first wavelength is emitted by a thulium laser.

22. The method of claim 20 wherein the second wavelength is emitted by an erbium laser.

23. The method of claim 22 wherein the second wavelength is approximately 2.94 μm.

24. The method of claim 20 wherein the first wavelength is emitted by a holmium laser.

25. The method of claim 20 wherein the second wavelength is emitted by a carbon dioxide laser.

26. The method of claim 20 further comprising viewing the bubble via optical coherence tomography.

27. The method of claim 20 wherein directing the first pulse of energy and the second pulse of energy to the region of tissue comprises directing the first pulse of energy and the second pulse of energy through a photonic crystal fiber.

28. The method of claim 20 wherein directing the first pulse of energy and the second pulse of energy to the region of tissue comprises directing the first pulse of energy and the second pulse of energy through a germanium dioxide fiber.

29. An apparatus comprising:
a first laser configured to direct a first pulse of energy at a first wavelength to a region of tissue;
a second laser configured to direct a second pulse of energy at a second wavelength to the region of tissue; and
a control system configured to control operation of the first laser and the second laser, wherein:
the control system is configured to control the duration of the first pulse of energy such that a vapor bubble is formed in the region of tissue after the first pulse of energy is directed to the region of tissue;
the control system is configured to control operation of the first laser and the second laser such that a delay period of time exists between the first pulse of energy and the second pulse of energy; and
the control system is configured to control the duration of the second pulse of energy such that the second pulse of energy breaks the mechanical integrity of the vapor bubble.

30. The apparatus of claim 29 wherein the first wavelength is emitted by a thulium laser.

31. The apparatus of claim 29 wherein the second wavelength is emitted by an erbium laser.

32. The apparatus of claim 31 wherein the second wavelength is approximately 2.94 μm.

33. The apparatus of claim 29 wherein the first wavelength is emitted by a holmium laser.

34. The apparatus of claim 29 wherein the second wavelength is emitted by a carbon dioxide laser.

35. The apparatus of claim 29 further comprising a conduit configured to direct the first pulse of energy and the second pulse of energy to the region of tissue.

36. The apparatus of claim 35 wherein the conduit comprises a photonic crystal fiber.

37. The apparatus of claim 35 wherein the conduit comprises a germanium dioxide fiber.

38. A method of ablating tissue, the method comprising:
directing energy from a ytterbium laser to provide pre-coagulation of blood vessels in a region of tissue to be resected;
directing energy from the ytterbium laser to increase the temperature of the region of tissue to be resected prior to resecting the tissue;
directing energy from a thulium laser to resect tissue from the region of tissue to be resected; and
directing energy from the thulium laser to provide post-resection coagulation.

39. The method of claim 38 further comprising a delay period between directing energy from the ytterbium laser to provide pre-coagulation of blood vessels in the region of tissue to be resected and directing energy from the ytterbium laser to increase the temperature of the region of tissue to be resected.
